(19) United States
(12) Patent Application Publication
(10) Pub. No.: US 2023/0169698 A1
(43) Pub. Date: Jun. 1, 2023

(54) MICROSCOPE SYSTEM AND CORRESPONDING SYSTEM, METHOD AND COMPUTER PROGRAM FOR A MICROSCOPE SYSTEM

(71) Applicants: Ohm SAVANAYANA, Au (CH); Leica Instruments (Singapore) Pte. Ltd., Singapore (SG)

(72) Inventor: Ohm SAVANAYANA, Au (CH)

(21) Appl. No.: 17/996,803

(22) PCT Filed: Apr. 20, 2021
(86) PCT No.: PCT/EP2021/060257

§ 371 Date: Oct. 21, 2022

(30) Foreign Application Priority Data
Apr. 24, 2020 (DE) 10 2020 111 220.3

(51) Int. Cl. G06T 11/00; G06F 3/041; G06F 3/0338

(52) U.S. Cl. CPC G06T 11/00; G06F 3/0416; G06F 3/0338; G06F 3/044

(57) ABSTRACT

Examples relate to a microscope system and to a corresponding system, method and computer program for a microscope system. The system comprises one or more processors and one or more storage devices. The system is configured to detect a presence of a finger of a user of the microscope at a control input device for controlling the microscope system. The system is configured to identify a control functionality associated with the control input device. The system is configured to generate a visual overlay based on the identified control functionality. The visual overlay comprises a representation of the control functionality. The system is configured to provide a display signal to a display device of the microscope system. The display signal comprises the visual overlay.

### TECHNICAL FIELD

Examples relate to a microscope system and to a corresponding system, method and computer program for a microscope system.

### BACKGROUND

Modern microscope systems, in particular surgical microscope systems, offer a wide variety of functionality to assist the user (i.e. surgeon) during operation of the microscope. At the same time, the user might prefer to keep their eyes at the eyepiece. For example, in surgical settings, the surgeon might prefer to keep looking at the surgical site to become quickly aware of bleeding. This may complicate the operation of the microscope system, as the input devices used to control the various functionalities may be occluded from the user.

### SUMMARY

There may be a desire for providing an improved concept for a microscope system, in which the functionality is made more easily accessible to the user of the microscope system.

This desire is addressed by the subject-matter of the independent claims.

Embodiments of the present disclosure provide a microscope system and a corresponding system, method and computer program for a microscope system. Embodiments of the present disclosure are based on the finding, that during the operation of microscopes, and in particular of surgical microscopes, the user/surgeon might not be able to take their eye off the sample/surgical site, e.g. to avoid overlooking the formation of bleeding in the wound tract. At the same time, the user/surgeon might prefer to use some of the additional functionality of the microscope system, such as a fluorescence mode, a recorder etc., which are usually accessible via input devices that are placed on the handles, or the case, of the respective (surgical) microscope, and which are occluded from the user while they are viewing the sample/surgical field through the oculars of the (surgical) microscope. Additionally, in many surgical microscopes, the control input devices are user-configurable, i.e. the user/surgeon may assign the respective functionality to the control input devices. If another user/surgeon uses such a customized microscope, they might not know the functionality of the respective control input device. Embodiments of the present disclosure thus provide a visual overlay that is overlaid over the view on the sample/surgical site being provided by the microscope, and which illustrates the control functionality being assigned to a control input device being touched (or close to being touched) by the user/surgeon of the microscope. A finger of the user of the microscope is detected at he control input device (i.e. in close proximity of the control input device, or touching the control input device), which is used to trigger the generation of the corresponding visual overlay.

Embodiments of the present disclosure provide a system for a microscope of a microscope system. The system comprises one or more processors and one or more storage devices. The system is configured to detect a presence of a finger of a user of the microscope at a control input device for controlling the microscope system. The system is configured to identify a control functionality associated with the control input device. The system is configured to generate a visual overlay based on the identified control functionality. The visual overlay comprises a representation of the control functionality. The system is configured to provide a display signal to a display device of the microscope system. The display signal comprises the visual overlay. By detecting the presence of the finger at the control input device, an imminent activation, or a desire to activate, the control input device may be detected. Based on said detection, the control functionality being associated with the control input device is presented to the user via the visual overlay, making the user aware of which functionality is about to be triggered.

In various embodiments, the system is configured to generate the visual overlay such, that the representation of the control functionality is shown while the presence of the finger at the control input device is detected. For example, the visual overlay may be generated such, that the representation of the control functionality is shown before the control input device is (fully) actuated. In other words, while the finger is at the control input device, the associated control functionality is shown, enabling the user to subsequently review the control functionality being provided via different control input devices.

In some embodiments, the system is configured to generate the visual overlay such, that the visual overlay further comprises an instruction for using the control functionality. Thus, for complex functionalities, additional guidance may be provided to the user.

The system may be configured to generate the visual overlay such, that the representation of the control functionality is partially overlaid by the display device over a view on a sample being provided by the microscope. Thus, both the view on the sample and the overlay may be visible at the same time.

For example, the control functionality may be a user-configurable control functionality. In these cases, the visual overlay may protect against accidental mis-use, as the control functionality being associated with a control input device may vary between microscope systems.

In various embodiments, the system is configured to obtain a sensor signal from a sensor of the microscope, and to detect the presence of the finger of the user based on the sensor signal. For example, the sensor signal may be indicative of the presence of the finger.

For example, the sensor signal may be a sensor signal of a capacitive sensor. For example, capacitive sensors may be used to detect the presence of a conductive object, such as a finger, in proximity of the capacitive sensor, and thus in proximity of the control input device, without actuating the control input device.

In some embodiments, the control input device is the sensor. For example, the control input device may be or comprise a capacitive sensor, or a control input facility being suitable for distinguishing between two actuation states (such as half-pressed and full-pressed) and may thus be suitable for distinguishing between a finger being present at the control input device, or a finger actuating the control input device.

Alternatively, the sensor may be separate from the control input device. For example, the control input device may be coupled with a capacitive sensor for detecting the presence of the finger.

A variety of types of control functionality may be associated with the control input device, and thus visualized for the user. For example, the control functionality may be one of a control functionality related to a magnification provided by the microscope, a control functionality related to a focusing functionality of the microscope, a control functionality related to a robotic arm of the microscope system, a control functionality related to a vertical or lateral movement of the microscope, a control functionality related to an activation of a fluorescence imaging functionality of the microscope, a control functionality related to a lighting functionality of the microscope system, a control functionality related to a camera recorder of the microscope system, a control function related to a head-up display of the microscope system, a control functionality related to an image-guided system, and control functionality related to an additional measurement facility (such as an endoscope or an optical coherence tomography functionality) of the microscope system.

In most cases, the microscope comprises both more than one functionality and, correspondingly, more than one control input devices. For example, the microscope may comprise a plurality of control input devices. Each control input device may be associated with a control functionality. The system may be configured to detect the presence of a finger of the user at a control input device of the plurality of control input devices, and to identify the control input device and the associated control functionality based on the detection of the finger. Thus, multiple control input devices may be distinguished in the generation of the visual overlay.

Embodiments of the present disclosure further provide a microscope system comprising the system, the microscope, the control input device and the display device. The system is configured to provide the display signal to the display device. While the term “microscope” refers to the optical carrier of the microscope system, the microscope system may comprise a multitude of devices, such as a robotic arm, an illumination system etc. For example, the microscope system may be a surgical microscope system.

For example, the display device may be one of an ocular display of the microscope, an auxiliary display of the surgical microscope system, and a headset display of the microscope system. For example, the proposed concept is applicable to different types of display devices of the microscope system.

In various embodiments, the control input device is occluded from the user of the microscope. The system may aid the user/surgeon in identifying the respective control input device.

There are various places, control input devices can be arranged at. For example, the control input device may be arranged (directly) at the microscope. Alternatively, the control input device is arranged at a handle of the microscope. Alternatively, the control input device may be a foot pedal of the microscope system

Also, different types of control devices may be used. For example, the control input device may be a button or a control stick.

Embodiments of the present disclosure further provide a method for a microscope system. The method comprises detecting a presence of a finger of a user of the microscope at a control input device for controlling the microscope system. The method comprises identifying a control functionality associated with the control input device. The method comprises generating a visual overlay based on the identified control functionality, the visual overlay comprising a representation of the control functionality. The method comprises providing a display signal to a display device of the microscope system, the display signal comprising the visual overlay.

Embodiments of the present disclosure further provide a computer program with a program code for performing the above method when the computer program is run on a processor.

### SHORT DESCRIPTION OF THE FIGURES

Some examples of apparatuses and/or methods will be described in the following by way of example only, and with reference to the accompanying figures, in which

FIG. 1a shows a block diagram of an embodiment of a system for a microscope of a microscope system;

FIG. 1b shows a block diagram of an embodiment of a surgical microscope system comprising a system;

FIGS. 1c and 1d show illustrations of exemplary visual overlays;

FIG. 2 shows a flow chart of a method for a microscope system; and

FIG. 3 shows a schematic diagram of a microscope system comprising a microscope and a computer system.

### DETAILED DESCRIPTION

Various examples will now be described more fully with reference to the accompanying drawings in which some examples are illustrated. In the figures, the thicknesses of lines, layers and/or regions may be exaggerated for clarity.

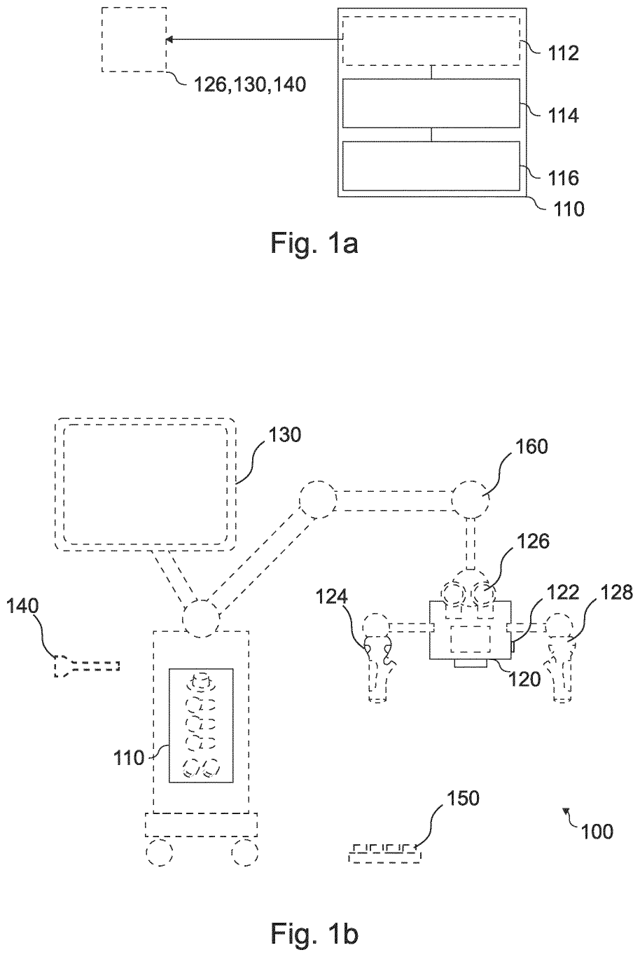

FIG. 1a shows a block diagram of an embodiment of a system 110 for a microscope 120 of a microscope system 120. The system 110 comprises one or more processors 114 and one or more storage devices 116. Optionally, the system further comprises an interface 112. The one or more processors 114 are coupled to the optional interface 112 and the one or more storage devices 116. In general, the functionality of the system 110 is provided by the one or more processors 114, e.g. in conjunction with the optional interface 112 and/or the one or more storage devices 116.

The system is configured to detect a presence of a finger of a user of the microscope at a control input device 122; 124; 150 that is suitable for controlling the microscope system 100. The system is configured to identify a control functionality associated with the control input device. The system is configured to generate a visual overlay based on the identified control functionality. The visual overlay comprises a representation of the control functionality. The system is configured to provide a display signal to a display device 126; 130; 140 of the microscope system (e.g. via the interface 112). The display signal comprises the visual overlay.

FIG. 1b shows a block diagram of microscope system 100, in particular of a surgical microscope system 100, comprising the system 110. The microscope system 100 further comprises the microscope 120, the control input device 122; 124; 150 and the display device 126; 130; 140. The system 110 is configured to provide the display signal to the display device. The microscope system shown in FIG. 1b is a surgical microscope system, which may be used at a surgical site by a surgeon. The surgical microscope system shown in FIG. 1b comprises a number of optional components, such as a base unit 105 (comprising the system 110) with a (rolling) stand, an auxiliary display 130, a (robotic or manual) arm 160 which holds the microscope 120 in place, and which is coupled to the base unit 105 and to the microscope 120, and steering handles 128 that are attached to the microscope 120. For example, the microscope 120 may comprise ocular eyepieces 126. The microscope system 100 may comprise a foot pedal (unit) 150, which may comprise one or more control input devices. In the context of this application, the term “(surgical) microscope system” is used, in order to cover the portions of the system that are not part of the actual microscope (which comprises optical components), but which are used in conjunction with the microscope, such as the display or a lighting system.

Embodiments of the present disclosure relate to a system, a method and a computer program that are suitable for a microscope system, such as the microscope system 100 introduced in connection with FIG. 1b. As has been introduced above, a distinction is made between the microscope 120 and the microscope system 100, with the microscope system comprising the microscope 120 and various components that are used in conjunction with the microscope 120, e.g. a lighting system, an auxiliary display etc. In a microscope system, the actual microscope is often also referred to as the “optical carrier”, as it comprises the optical components of the microscope system. In general, a microscope is an optical instrument that is suitable for examining objects that are too small to be examined by the human eye (alone). For example, a microscope may provide an optical magnification of an object. In modern microscopes, the optical magnification is often provided for a camera or an imaging sensor. The microscope 120 may further comprise one or more optical magnification components that are used to magnify a view on the sample.

There are a variety of different types of microscopes. If the microscope system is used in the medical or biological fields, the object being viewed through the microscope may be a sample of organic tissue, e.g. arranged within a petri dish or present in a part of a body of a patient. For example, the microscope system 100 may be a microscope system for use in a laboratory, e.g. a microscope that may be used to examine the sample of organic tissue in a petri dish. Alternatively, the microscope 120 may be part of a surgical microscope system 100, e.g. a microscope to be used during a surgical procedure. Such a system is shown in FIG. 1b, for example. Although embodiments are described in connection with a microscope system, they may also be applied, in a more general manner, to any optical device. For example, the microscope system may be a system for performing material testing or integrity testing of materials, e.g. of metals or composite materials.

The system is configured to detect the presence of a finger of the user of the microscope at a control input device 122; 124; 150 for controlling the microscope system 100. In general, a control input device of the microscope system 100 is an input device, such as a button, foot pedal or control stick, that is suitable for, or rather configured to, control the microscope system, e.g. the microscope 120, or one of the other components of the microscope system 100. In other words, the control input device may be configured to control a functionality of the microscope system or microscope 120. Accordingly, the control input device is associated with a control functionality of the microscope system 100. Most microscope systems may comprise more than one control input device. For example, the microscope system may comprise different functionalities, wherein each of the different functionalities (or at least a subset of the different functionalities) is associated with one control input device, i.e. wherein each of the different functionalities (or at least a subset of the different functionalities) is controlled by one control input devices. Accordingly, the microscope may comprise a plurality of control input devices, with each control input device being associated with a (specific) control functionality. For example, there may be a 1-to-1 association between control input device and control functionality.

In general, the concepts is applicable to different types of control input devices of the microscope system, and also to various placements of the control input devices. In many cases, the microscopes are being used via ocular eyepieces, or via a headset display. Additionally or alternatively, the control input device(s) may be placed at the backside, or at a foot pedal of the microscope 120 or microscope system 100. Consequently, the control input device(s) may be occluded from the user of the microscope (during usage of the microscope 120 by the user), e.g. while the user uses the ocular eyepieces or the headset display, or due to the placement of the control input device(s) at the far side of the microscope system while the user is using the microscope system. In such cases, it may be especially useful to get feedback on the functionality being associated with the respective control input device.

In general, the control input device(s) may be arranged at different parts of the microscope system. For example, the control input device 122 (e.g. one or more of the control input devices) may be arranged (directly) at the microscope 120, e.g. at the body of the microscope 120. In some embodiments, the microscope system may also comprise steering handles 128, which are often arranged at the microscope 120, enabling the user to move the microscope 120 relative to the sample/patient. For example, the microscope 120 may be held in place by a manual or robotic arm, and the handles 128 may be used to move the microscope 120 that is suspended from the manual or robotic arm. The control input device(s) (e.g. one or more of the control input devices) 124 may be arranged at a (or both) handle 128 of the microscope 120. Alternatively or additionally, one or more of the control input devices may be foot pedals of the microscope system 120. In other words, the control input device may be a foot pedal 150 of the microscope system 120.

There are various types of control input devices. For example, the control input device (or a control input device of the plurality of control input devices) may be a button, such as haptic button or a capacitive button. For example, a haptic button may be actuated by displacing the button from a first position (e.g. a resting position) to a second position (e.g. an actuation position). A capacitive button may be actuated by touching the capacitive button (e.g. without moving the button, as the capacitive button is a static sensor). Alternatively or additionally, the control input device (or a control input device of the plurality of control input devices) may be a control stick (i.e. a joystick) or four-way/eight-way controller, which is a controller that can be used to select one of four/eight directions.

In various embodiments, the presence of the finger can be detected using a sensor of the microscope system. For example, the system may be configured to obtain a sensor signal from a sensor 122; 124; 150 of the microscope (e.g. via the interface 112), and to detect the presence of the finger of the user based on the sensor signal. In other words, the system may be configured to detect the presence of the finger using a sensor. In various embodiments, the respective control input device may be the sensor. Alternatively, the sensor may be separate from the control input device. In this case, for example, the sensor may be a capacitive sensor that is arranged at the control input device, or that is integrated within a portion of the control input device (without acting as trigger for the control function). For example, the control input device may be a touch sensor (e.g. a capacitive (touch) sensor), and the system may be configured to detect the presence of the finger at the control input device via the touch sensor. For example, in capacitive sensors, a distinction can be made between the presence of a conductive object (such as the finger) and force being applied to the capacitive sensors (i.e. an actuation of the sensor), e.g. based on a difference between the capacitive values being measured in both cases. For example, the sensor signal may be a sensor signal of a capacitive sensor. Accordingly, the sensor signal may be indicative of the presence of the finger, or indicative of force being applied to the capacitive sensors. The system may be configured to distinguish between the presence of the finger and the actuation of the sensor based on the sensor data. In some embodiments, the control input device may be a control input facility being suitable for distinguishing between two actuation states (such as half-pressed and fully pressed, partial actuation and full actuation), e.g. similar to a shutter button of a camera, which triggers the auto-focus when half-pressed and the shutter when fully pressed. For example, the system may be configured to distinguish between a partial actuation (being indicative of the presence of the finger) and the full actuation (triggering the control function) based on the sensor signal. In other words, the system may be configured to differentiate between the presence of the finger at the sensor and the actuation of the sensor, or between two a partial actuation and a full actuation of the sensor, to detect the presence of the finger at the sensor.

The system is configured to identify the control functionality associated with the control input device. As has been pointed out before, the (or each) control input device may be associated with a (e.g. one specific) control functionality. The association between control input device(s) and control functionality (or functionalities) may be stored in a data structure, which may be stored using the one or more storage devices. The system may be configured to determine the control functionality associated with the control input device based on the data structure and based on the control input device the presence of the finger is detected at. As has been pointed out before, the microscope system may have both a plurality of control input devices and a plurality of control functionalities. Each control input device (of the plurality of control input devices) may be associated with a control functionality (of the plurality of control functionalities). The system may be configured to detect the presence of a finger of the user at a control input device of the plurality of control input devices, and to identify the control input device and the associated control functionality based on the detection of the finger. In other words, the system may be configured to identify the control input device at which the finger is detected (e.g. based on the sensor signal), and to identify the associated control functionality based on the control input devices the finger is detected at.

In general, there are a multitude of different control functionalities that can be implemented by a modern (surgical) microscope system. For example, the control functionality may be one of, or the plurality of control functionalities may comprise one or more of, a control functionality related to a magnification provided by the microscope, a control functionality related to a focusing functionality of the microscope, a control functionality related to a robotic arm 160 of the microscope system, a control functionality related to a vertical or lateral movement of the microscope, a control functionality related to an activation of a fluorescence imaging functionality of the microscope, a control functionality related to a lighting functionality of the microscope system, a control functionality related to a camera recorder of the microscope system, a control function related to a head-up display of the microscope system, a control functionality related to an image-guided system, and control functionality related to an additional measurement facility (such as an optical coherence tomography, OCT, sensor or an endoscope) of the microscope system. Due to personal preference, or applicability to a specific kind of operation of the microscope (e.g. due to a specific type of surgical procedure being performed with the help of the surgical microscope), different control functionalities may be mapped to the control input device(s). Accordingly, the control functionality may be a user-configurable control functionality, i.e. at least a subset of the plurality of control functionalities may be user-configurable control functionalities. In other words, the association between a control input device and a control functionality may be configured or configurable by a user of the microscope system.

The system is configured to generate the visual overlay based on the identified control functionality, with the visual overlay comprising a representation of the control functionality. In general, the visual overlay may satisfy two criteria—it may provide a representation of the identified control functionality, and, at the same time, it might not (overly) obstruct the view on the sample being provided by the microscope. For example, the system may be configured to generate the visual overlay such, that the representation of the control functionality is partially overlaid by the display device 130 over a view on a sample (e.g. a surgical site) being provided by the microscope 120. For example, the visual overlay may be generated such, that the representation of the identified control functionality is shown at the periphery of the view on the sample being provided by the microscope 120. Examples are shown in FIGS. 1c and 1d. FIGS. 1c and 1d show illustrations of exemplary visual overlays. In FIGS. 1c and 1d, the view through two eyepieces 170a; 170b (left and right) is shown. In general, there are different feasible representations. For example, the representation may be a graphical representation (such as an icon representing the control functionality) or a textual representation (e.g. a name of the control functionality) of the control functionality. For example, in FIG. 1c, a graphical/icon representation 172 is shown, along with a horizontal textual representation 174 and a vertical textual representation 176. At the same time, only one representation, or multiple representations of the same control functionality might be shown. For example, both a graphical representation and a textual representation of the same control functionality might be shown at the same time. In some embodiments, the system is configured to generate the visual overlay such, that the visual overlay further comprises an instruction for using the control functionality. For example, in FIG. 1d, information/an instruction on using the control functionality 178 is shown on the bottom of the view, in addition to the textual representation (or any representation) of the control functionality at the top of the view. In general, the visual overlay may be generated such, that both the representation and the instruction for using the control functionality are shown at the periphery of the view. For example, the visual overlay (i.e. the representation of the control functionality and the instruction for using the control functionality) may overlay at most 25% (or at most 20%, at most 15%, at most 10%) of the view on the sample.

In embodiments, the visual overlay may be shown when it is desired by the respective user, e.g. when the user is about the activate a control functionality of the microscope system. There are (at least) two general approaches for achieving this—by continuously generating the visual overlay, and blinding out/fading down the visual overlay while it is not desired, or by generating the visual overlay (only) when it is desired. For example, the system may be configured to continuously generate the visual overlay, with the visually overlay being devoid of the representation of the control functionality when the presence of the finger is not detected. Alternatively, the system may be configured to generate the visual overlay in response to the detection of the presence of the finger. In any case, the representation (and the respective instructions) may be shown as long as the presence of the finger is detected (or the control input device is actuated). For example, the system may be configured to generate the visual overlay such, that the representation of the control functionality is shown while (e.g. as long as) the presence of the finger at the control input device is detected. For example, the presence of the finger at the control input device may be deemed detected while the control input device is being actuated. For example, the visual overlay may be shown before the respective control input device is (fully) actuated.

The system is configured to provide a display signal to a display device 126; 130; 140 of the microscope system (e.g. via the interface 112), the display signal comprising the visual overlay. The display device may be configured to show the visual overlay based on the display signal, e.g. to inject the visual overlay over the view on the sample based on the display signal. For example, the display signal may comprise a video stream or control instructions that comprise the visual overlay, e.g. such that the visual overlay is shown by the respective display device. For example, the display device may be one of an ocular display 126 of the microscope, an auxiliary display 130 of the surgical microscope system, and a headset display 140 of the microscope system. In modern (surgical) microscope systems, the view on the sample is often provided via a display, such as a ocular display, an auxiliary display or a headset display, e.g. using video stream that is generated based on image sensor data of an optical imaging sensor of the respective microscope. In this case, the visual overlay may be merely overlaid over the video stream. For example, the system may be configured to obtain image sensor data of an optical imaging sensor of the microscope, to generate the video stream based on the image sensor data and to generate the display signal by overlaying the visual overlay over the video stream.

Alternatively, the visual overlay may be overlaid over an optical view of the sample. For example, the ocular eyepieces of the microscope may be configured to provide an optical view on the sample, and the display device may be configured to inject the overlay into the optical view on the sample, e.g. using a one-way mirror or a semi-transparent display that is arranged within an optical path of the microscope. For example, the microscope may be an optical microscope with at least one optical path. One-way mirror(s) may be arranged within the optical path(s), and the visual overlay may be projection onto the one-way mirror(s) and thus overlaid over the view on the sample. In this case, the display device may be a projection device configured to project the visual overlay towards the mirror(s), e.g. such that the visual overlay is reflected towards an eyepiece of the microscope. Alternatively, a display or displays may be used to provide the overlay within the optical path(s) of the microscope. For example, the display device may comprise at least one display being arranged within the optical path(s). For example, the display(s) may be one of a projection-based display and a screen-based display, such as a Liquid Crystal Display (LCD)—or an Organic Light Emitting Diode (OLED)-based display. For example, the display(s) may be arranged within the eyepiece of the optical stereoscopic microscope, e.g. one display in each of the oculars. For example, two displays may be used to turn the oculars of the optical microscope into augmented reality oculars, i.e. an augmented reality eyepiece. Alternatively, other technologies may be used to implement the augmented reality eyepiece/oculars.

The interface 112 may correspond to one or more inputs and/or outputs for receiving and/or transmitting information, which may be in digital (bit) values according to a specified code, within a module, between modules or between modules of different entities. For example, the interface 112 may comprise interface circuitry configured to receive and/or transmit information. In embodiments the one or more processors 114 may be implemented using one or more processing units, one or more processing devices, any means for processing, such as a processor, a computer or a programmable hardware component being operable with accordingly adapted software. In other words, the described function of the one or more processors 114 may as well be implemented in software, which is then executed on one or more programmable hardware components. Such hardware components may comprise a general-purpose processor, a Digital Signal Processor (DSP), a micro-controller, etc. In at least some embodiments, the one or more storage devices 116 may comprise at least one element of the group of a computer readable storage medium, such as an magnetic or optical storage medium, e.g. a hard disk drive, a flash memory, Floppy-Disk, Random Access Memory (RAM), Programmable Read Only Memory (PROM), Erasable Programmable Read Only Memory (EPROM), an Electronically Erasable Programmable Read Only Memory (EEPROM), or a network storage.

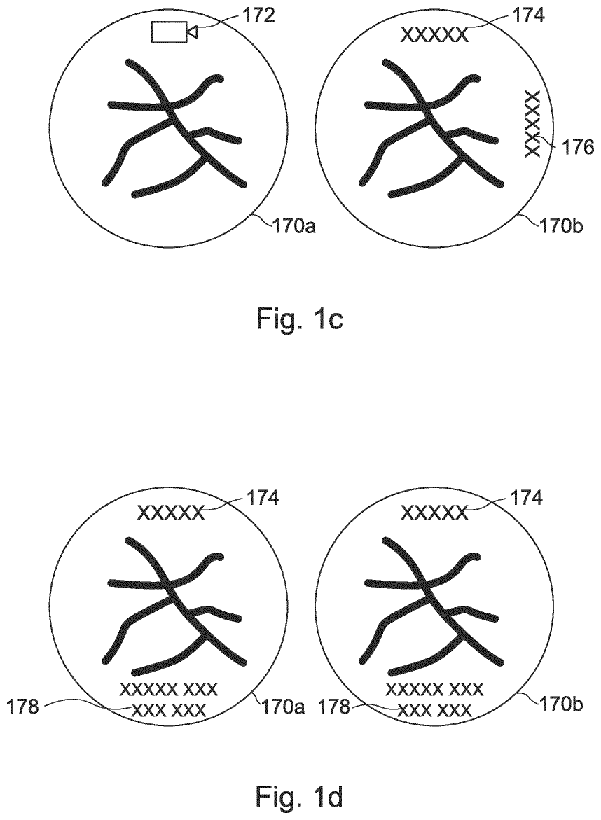

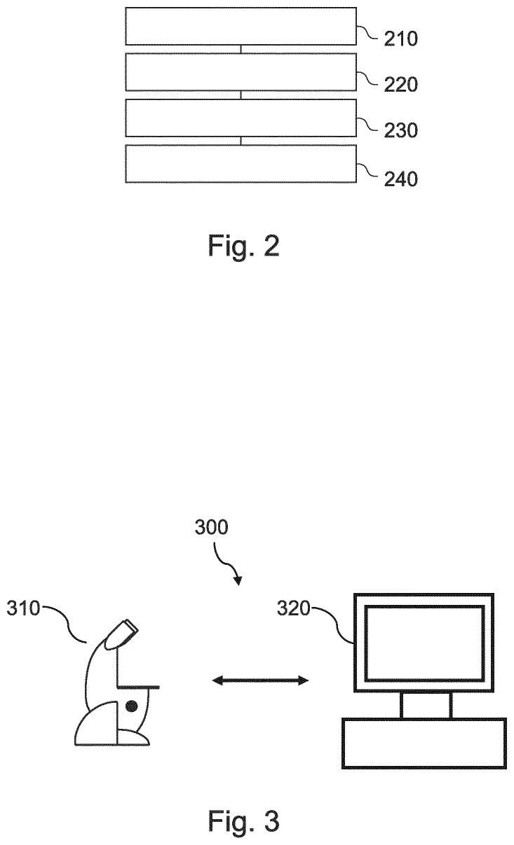

More details and aspects of the system and microscope system are mentioned in connection with the proposed concept or one or more examples described above or below (e.g. FIG. 2 or 3). The system or microscope system may comprise one or more additional optional features corresponding to one or more aspects of the proposed concept or one or more examples described above or below.

FIG. 2 shows a flow chart of an embodiment of a (corresponding) method for a microscope system 100. The method comprises detecting 210 a presence of a finger of a user of the microscope at a control input device 122; 124; 150 for controlling the microscope system 100. The method comprises identifying 220 a control functionality associated with the control input device. The method comprises generating 230 a visual overlay based on the identified control functionality. The visual overlay comprises a representation of the control functionality. The method comprises providing 240 a display signal to a display device 126; 130; 140 of the microscope system, the display signal comprising the visual overlay. For example, the method may be performed by the microscope system 100, e.g. by the system 110 of the microscope system.

As indicated above, features described in connection with the system 110 and the microscope system 100 of FIGS. 1a and/or 1b may be likewise applied to the method of FIG. 2.

More details and aspects of the method are mentioned in connection with the proposed concept or one or more examples described above or below (e.g. FIG. 1a to 1d, 3). The method may comprise one or more additional optional features corresponding to one or more aspects of the proposed concept or one or more examples described above or below.

Various embodiments of the present disclosure relate to function recognition and to a display of the recognized function, i.e. to a function display. Embodiments of the present disclosure may be based on the user of sensors, such as touch sensors, and based on the display of graphical image on a display device, such as eyepieces, via image injection or via a monitor/display.

In some high-end vehicles, a guidance function is offered to guide the user in the electric adjustment of their seat. When the driver touches the seat adjustment functions on the side of the seat, the function is displayed on the main display of the vehicle, which may be useful, as the seat adjustment input device is usually occluded from the user, making it impossible for the user to actually see a label of the input device.

In microsurgery, a similar scenario may present itself. During surgery, when the surgeon wants to change function (s) of the microscope by using buttons on the handgrips or footswitch, they might not know what function they are activating, e.g. due to a large number of different buttons, or due to the configurability of the microscope.

In embodiments, when the button equipped with sensor on the handgrips or footswitch is touched by the surgeon, the label of the function e.g. zoom, focus, use of an image processing overlay to highlight portions of the surgical site, may be graphically displayed via image injection in the ocular and/or on the screen.

To give an example, the surgeon would like to activate zoom function to increase the magnification on the surgical microscope, e.g. within an augmented reality- or image-guided functionality of the surgical microscope. While looking through the eyepieces at the surgical field, they (i.e. the surgeon) may reach up with their hand to the handgrip of the microscope and touch the button, which they believe would activate the zoom function. The touch-sensor on the button may send the signal to the microscope command processing unit (e.g. the system 110 of the microscope system). The command processing unit may activate the function to graphically display the button with label zoom or other function e.g. focus in the surgeon's eyepiece via image injection or via a display. The surgeon may be informed whether they are about to activate the zoom function.

More details and aspects of the microscope system are mentioned in connection with the proposed concept or one or more examples described above or below (e.g. FIG. 1a to 2, 3). The microscope system may comprise one or more additional optional features corresponding to one or more aspects of the proposed concept or one or more examples described above or below.

Some embodiments relate to a microscope comprising a system as described in connection with one or more of the FIGS. 1 to 2. Alternatively, a microscope may be part of or connected to a system as described in connection with one or more of the FIGS. 1 to 2. FIG. 3 shows a schematic illustration of a system 300 configured to perform a method described herein. The system 300 comprises a microscope 310 and a computer system 320. The microscope 310 is configured to take images and is connected to the computer system 320. The computer system 320 is configured to execute at least a part of a method described herein. The computer system 320 may be configured to execute a machine learning algorithm. The computer system 320 and microscope 310 may be separate entities but can also be integrated together in one common housing. The computer system 320 may be part of a central processing system of the microscope 310 and/or the computer system 320 may be part of a subcomponent of the microscope 310, such as a sensor, an actor, a camera or an illumination unit, etc. of the microscope 310.

The computer system 320 may be a local computer device (e.g. personal computer, laptop, tablet computer or mobile phone) with one or more processors and one or more storage devices or may be a distributed computer system (e.g. a cloud computing system with one or more processors and one or more storage devices distributed at various locations, for example, at a local client and/or one or more remote server farms and/or data centers). The computer system 320 may comprise any circuit or combination of circuits. In one embodiment, the computer system 320 may include one or more processors which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA), for example, of a microscope or a microscope component (e.g. camera) or any other type of processor or processing circuit. Other types of circuits that may be included in the computer system 320 may be a custom circuit, an application-specific integrated circuit (ASIC), or the like, such as, for example, one or more circuits (such as a communication circuit) for use in wireless devices like mobile telephones, tablet computers, laptop computers, two-way radios, and similar electronic systems. The computer system 320 may include one or more storage devices, which may include one or more memory elements suitable to the particular application, such as a main memory in the form of random access memory (RAM), one or more hard drives, and/or one or more drives that handle removable media such as compact disks (CD), flash memory cards, digital video disk (DVD), and the like. The computer system 320 may also include a display device, one or more speakers, and a keyboard and/or controller, which can include a mouse, trackball, touch screen, voice-recognition device, or any other device that permits a system user to input information into and receive information from the computer system 320.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may, for example, be stored on a machine readable carrier.

Other embodiments comprise the computer program for performing one of the methods described herein, stored on a machine readable carrier.

In other words, an embodiment of the present invention is, therefore, a computer program having a program code for performing one of the methods described herein, when the computer program runs on a computer.

A further embodiment of the present invention is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present invention is an apparatus as described herein comprising a processor and the storage medium.

A further embodiment of the invention is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

### LIST OF REFERENCE SIGNS

100 Microscope system
105 Base unit
110 System
112 Interface
114 One or more processors
116 one or more storage devices
120 Microscope
122 Control input device
124 Control input device
126 Ocular display
128 Handles
130 Auxiliary display
140 Headset display
150 Foot pedal
160 Robotic arm
170a View through left eyepiece
170b View through left eyepiece
172 Icon representation
174 Horizontal textual representation
176 Vertical textual representation
178 Instruction on using the control functionality
210 Detecting the presence of a finger
220 Identifying a control function
230 Generating a visual overlay
240 Providing a display signal
300 System
310 Microscope
320 Computer system

1. A system for a microscope of a microscope system, the system comprising one or more processors and one or more storage devices, wherein the system is configured to:
detect a presence of a finger of a user of the microscope at a control input device for controlling the microscope system;
identify a control functionality associated with the control input device;
generate a visual overlay based on the identified control functionality, the visual overlay comprising a representation of the control functionality; and
provide a display signal to a display device of the microscope system, the display signal comprising the visual overlay.

2. The system according to claim 1, wherein the control input device is one of a button and a control stick.

3. The system according to claim 1, wherein the system is configured to generate the visual overlay in response to the detection of the presence of the finger.

4. The system according to claim 1, wherein the system is configured to generate the visual overlay such, that the representation of the control functionality is shown while the presence of the finger at the control input device is detected.

5. The system according to claim 1,
wherein the system is configured to generate the visual overlay such, that the visual overlay further comprises an instruction for using the control functionality, and/or wherein the system is configured to generate the visual overlay such, that the representation of the control functionality is partially overlaid by the display device over a view on a sample being provided by the microscope.

6. The system according to claim 1, wherein the control functionality is a user-configurable control functionality.

7. The system according to claim 1, wherein the system is configured to obtain a sensor signal from a sensor of the microscope, and to detect the presence of the finger of the user based on the sensor signal.

8. The system according to claim 7,
wherein the sensor signal is a sensor signal of a capacitive sensor.

9. The system according to claim 7, wherein the system is configured to differentiate between the presence of the finger at the sensor and the actuation of the sensor, or between two a partial actuation and a full actuation of the sensor, to detect the presence of the finger at the sensor.

10. The system according to claim 1, wherein the microscope comprises a plurality of control input devices, wherein each control input device is associated with a control functionality, wherein the system is configured to detect the presence of a finger of the user at a control input device of the plurality of control input devices, and to identify the control input device and the associated control functionality based on the detection of the finger.

11. A microscope system comprising the system according to claim 1, the microscope, the control input device and the display device, wherein the system is configured to provide the display signal to the display device.

12. The microscope system according to claim 11, wherein the control input device is occluded from the user of the microscope.

13. The microscope system according to claim 11, wherein the microscope system is a surgical microscope system.

14. A method for a microscope system, the method comprising:
detecting a presence of a finger of a user of a microscope of the microscope system at a control input device for controlling the microscope system;
identifying a control functionality associated with the control input device;
generating a visual overlay based on the identified control functionality, the visual overlay comprising a representation of the control functionality; and
providing a display signal to a display device of the microscope system, the display signal comprising the visual overlay.

15. A non-transitory, computer-readable medium comprising a program code that, when the program code is executed on a processor, a computer, or a programmable hardware component, causes the processor, the computer, or the programmable hardware component to perform the method of claim 14.

16. The system according to claim 1, wherein the control input device is occluded from the user of the microscope during usage of the microscope by the user.

17. The system according to claim 8, wherein the control input device is the sensor or wherein the sensor is separate from the control input device.

18. The microscope system according to claim 11, wherein the control input device is arranged at the microscope.

19. The microscope system according to claim 11, wherein the control input device is arranged at a handle of the microscope.

20. The microscope system according to claim 11, wherein the control input device is one of a button or a control stick.
